(19) United States
(12) Patent Application Publication
(10) Pub. No.: US 2011/0256624 A1
(43) Pub. Date: Oct. 20, 2011

(54) BIOREACTOR

(75) Inventors: Marc Jenne, Leverkusen (DE); Björn Frahm, Lemgo (DE); Joerg Kauling, Bergisch Gladbach (DE); Helmut Brod, koln (DE)

(73) Assignee: BAYER TECHNOLOGY SERVICES GMBH, LEVERKUSEN (DE)

(21) Appl. No.: 13/140,464

(22) PCT Filed: Dec. 8, 2009
(86) PCT No.: PCT/EP09/08733

§ 371 Date: Jun. 17, 2011

(30) Foreign Application Priority Data
Dec. 20, 2008 (DE) 10 2008 064 279.7

(51) Int. Cl. C12M 1/04; C12N 5/071; C12N 1/00; C12N 5/07; C12N 5/04

(52) U.S. Cl. 435/366; 435/296.1; 435/325; 435/420; 435/243

(57) ABSTRACT

The invention relates to a bioreactor, to the use of the bioreactor for culturing microorganisms or cell cultures, and also to a method for culturing microorganisms or cell cultures.

The invention relates to a bioreactor, the use of the bioreactor for culturing microorganisms or cell cultures, and also to a method for culturing microorganisms or cell cultures.

In the culturing of microorganisms and cell cultures, in particular of animal, plant and human cells, various types of bioreactors are used. In addition to the stirred bioreactor, above all the air-lift bioreactor has become established. In an air-lift bioreactor, gas such as, for example, air, is introduced into an upwardly directed part of the bioreactor, in the speciality also known as a riser. Preferably, gas introduction takes place as fine bubbles. The riser is connected at its top and bottom end to the top and bottom end of a further upwardly directed part of the bioreactor, known in the speciality as a downcomer. A widespread variant of the substantially cylindrical air-lift bioreactor contains a centrally arranged cylindrical guide tube which divides the air-lift bioreactor into an ascending part (riser) within the guide tube and a descending part (downcomer) in the annular space between the guide tube and the vessel outer wall of the air-lift bioreactor. The ascending part can equally well be situated in the annular space between the guide tube and the vessel outer wall and the descending part within the guide tube. The feed of, for example, oxygen-enriched gas at the bottom end of the riser decreases the median density of the suspension culture in the riser, which leads to an upwardly directed liquid flow in the riser, which consequently replaces the liquid contents of the downcomer, which in turn flow to the bottom end of the riser. In this manner a liquid circulation is generated which mixes the suspension culture sufficiently and retains the cells in suspension, i.e. in free suspension. In the case of cells having an oxygen requirement, gaseous oxygen dissolves, for example, in the nutrient medium and is used in respiration to form carbon dioxide by the cells present in the suspension culture. The advantage of such a “stirred” bioreactor is that with sufficient supply of the cells with oxygen dissolved in the nutrient medium and sufficient disposal of the carbon dioxide formed in the respiration, no moving parts such as a mechanical agitator are necessary.

The air-lift bioreactors known in the prior art are designed in a slender construction, i.e. the ratio H/D of height H to diameter D, in the air-lift bioreactors known in cell culture, is between 6 and 14:
[1] Varley J., Birch J., Reactor design for large scale suspension animal cell culture, Cytotechnology, 29, (1999): 177-205.
[2] Petrossian A., Cortessis G. P., Large-scale production of monoclonal antibodies in defined serum-free media in airlift bioreactors, BioTechniques, 8, (1990): 414-422.
[3] Hesse F., Ebel M., Konisch N., Sterlinski R., Kessler W., and Wagner R., Comparison of a production process in a membrane-aerated stirred tank and up to 1000-L airlift bioreactors using BHK-21 cells and chemically defined protein-free medium, Biotechnol. Prog., 19, 3 (2003): 833-843.
[4] Chisti, Y., Animal-cell damage in sparged bioreactors, Trends Biotechnol., 18, 10 (2000): 420-432

On a production scale, this slender construction leads to the air-lift bioreactors reaching heights of several metres for customary working volumes of several hundred litres to several cubic metres. For example, 12 m³ of working volume is equivalent to a height of 14.4 m at an H/D ratio of 14. Such air-lift bioreactors must therefore be erected in rooms having high ceilings or with breakthroughs over several stories. This requires a complex steel frame construction. Furthermore, the air-lift bioreactors must be steam-sterilized in situ and can no longer be steam-sterilized as a whole in an autoclave together with the peripherals necessary for cell culture. Conventional bioreactors having current H/D ratios around 2 can, in contrast, be transported into autoclaves and steam-sterilized there.

In general, high reactors are difficult to handle.

Proceeding from the prior art, the object is therefore to provide bioreactors which, even in the case of working volumes of several hundred litres up to several cubic metres, keep within heights which correspond to customary room heights, and so rebuilding measures for the installation are not necessary. In this process, the bioreactors required shall have, like the air-lift bioreactors known from the prior art, sufficient supply of cells with gas, e.g. oxygen, and sufficient disposal of gas, e.g. the carbon dioxide formed in the respiration, without moving parts such as a mechanical agitator being required.

Surprisingly, it has been found that air-lift bioreactors can be used in cell culture even if the ratio of height to diameter is markedly below 6.

The present invention therefore relates to an air-lift bioreactor having a ratio H/D of height H to diameter D which is less than 6.

Preferably, the ratio H/D is between 1 and 6, particularly preferably between 2 and 6.

An air-lift bioreactor is taken to mean a reactor which possesses a riser, a downcomer and a gas-introduction unit.

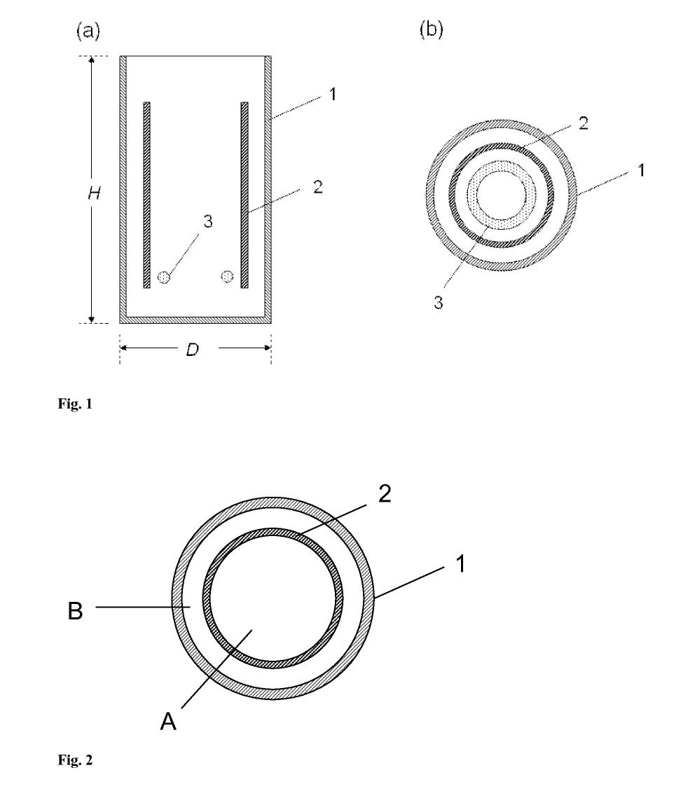

Riser and downcomer are preferably formed by a cylindrical vessel into which a cylindrical tube is arranged (see, e.g., FIG. 1). In a preferred embodiment, the cross sectional areas of the riser and the downcomer differ by a maximum of 10%, particularly preferably they are equal (see, e.g., FIG. 2).

The cylindrical vessel and the cylindrical tube preferably have the same cross sectional geometry. They are preferably constructed to elliptical or round.

The gas-introduction unit is either arranged within the cylindrical guide tube or between outer wall of the guide tube and inner wall of the vessel. In the first case, the riser is within the guide tube and the downcomer between outer wall of the guide tube and inner wall of the vessel; in the second case, the downcomer is within the guide tube and the riser between outer wall of the guide tube and inner wall of the vessel.

In addition to supplying the cells or organisms with gas, e.g. oxygen, and transporting away gaseous metabolic products such as, e.g., carbon dioxide, the gas-introduction unit effects a circulation flow between riser and downcomer.

Preferably, a gas-introduction unit is used which generates bubbles having a diameter of less than 2 mm

In a particularly preferred embodiment, the gas-introduction unit is constructed as a microbubble sparger. Microbubble spargers are taken to mean bodies which can introduce gas, in particular oxygen, in the form of fine bubbles into a liquid. “Fine gas bubbles” are taken to mean gas bubbles which have a small tendency to coalesce in the culture medium used. Suitable microbubble spargers are, for example, special sintered bodies made of metal or ceramic materials, filter plates, or laser-perforated plates which have pores or holes having a diameter of generally less than 100 μm, preferably 15 μm. The gas-introduction unit is preferably constructed as a hollow body, e.g. as a tube, through which gas can flow. At low gas superficial velocities of less than 0.5 m h⁻¹, very fine gas bubbles are generated which have a low tendency to coalesce in the media usually used in cell culture.

Suitable microbubble spargers are, in addition, flexible membrane tubes. Flexible membrane tubes are taken to mean flexible tubular structures which are permeable to gases such as oxygen and carbon dioxide. As an example, hollow filament membranes made of microporous polypropylene may be mentioned, as are described, for example, in Chem.-Ing.-Tech. 62 (1990), No. 5, pp. 393-395 by H. Büintemeyer et al.

The gas-introduction unit is preferably arranged close to the lower rim of the guide tube. The gas-introduction unit is preferably constructed to be ring-shaped or spiral, so that it does not significantly decrease the flow cross section. Plate-shaped gas-introduction units lead to an increased resistance to flow. The resultant pressure drop must be compensated for by a higher gas volumetric flow rate in order to maintain the circulation flow between riser and downcomer. A higher gas volumetric flow rate, however, leads to an increased shearing rate which can be destructive to sensitive cells and should therefore be avoided. In addition, the diameter of the preferably ring-shaped or spiral gas-introduction unit should be shaped to fit the cross section of the riser in such a manner that the cross section is charged with gas bubbles as uniformly as possible. Therefore, a gas-introduction unit should be avoided which is arranged with a small ring-shaped diameter in the centre of the riser, wherein the residual (outer) riser cross section is inadequately supplied with the resultant gas bubbles. It is also conceivable to construct the gas-introduction unit in a meander shape. Further shapes are conceivable.

In a preferred embodiment, all corners and edges within the bioreactor according to the invention are rounded off, in particular the edges of the guide tube, in order to avoid eddies which likewise lead to a pressure drop and increased shear.

The bioreactor according to the invention preferably has means for conducting flow which promote a loop flow between riser and downcomer, and also keep pressure drops and shearing low. In a preferred embodiment, the bottom of the bioreactor has an elevation which deflects upwards the liquid flowing to the reactor bottom. Preferably, the flow cross sections in the lower and upper region of the bioreactor, in which the deflection of the direction of flow takes place and the medium transfers from the riser to the downcomer or from the downcomer to the riser, are equal and correspond in their size to the flow cross sections of the riser and downcomer.

Suitable material for the guide tube and the vessel are the materials customarily used in biotechnology for culturing microorganisms and cells, such as, e.g., VA steel or glass.

The guide tube is held within the vessel via supports. These can be mounted on the bottom of the vessel, on the lid of the vessel, or on the inner wall of the vessel. In a preferred embodiment, the guide tube is suspended on supports which are mounted on the lid of the vessel. Via the lid, the bioreactor is customarily supplied with medium, nutrients, additions (such as, e.g., antifoams and buffers) and gases.

The bioreactor according to the invention is suitable for culturing microorganisms and cells (plant, animal, human) of all types. The use of the bioreactor according to the invention for culturing microorganisms or plant, animal or human cells is subject matter of the present invention.

The present invention further relates to a method for culturing microorganisms or cell cultures. The method is characterized in that, in a bioreactor having a ratio H/D of height H to diameter D less than 6, preferably between 2 and 6, a loop flow (circulation flow) between an inner guide tube and the region between the outer wall of the guide tube and the inner wall of the bioreactor is generated by means of a gas-introduction unit. The gas-introduction unit is preferably a unit which generates bubbles having a diameter of less than 2 mm, particularly preferably, the unit is a microbubble sparger.

The gas volumetric flow rate in this case is selected in such a manner that the loop flow is maintained and the cells are adequately supplied with gas, e.g. oxygen, and are freed from unwanted gas, e.g. carbon dioxide, but the shearing rates are kept minimal in order to avoid destruction of sensitive cells. In addition, the gas volumetric flow rate is selected in such a manner that suspension of the cells is ensured, and sedimentation is therefore prevented. Further (subsidiary) criteria are a sufficiently short mixing time and foam formation as low as possible.

The gas bubbles can lead to the formation of foam. However, foam formation must be avoided since cells have a tendency to float with the foam. In the foam layer there are inadequate culture conditions. The use of antifoams can, as is known, provide a remedy here.

Preferably, the method according to the invention is operated in such a manner that the shell areas above and below the guide tube differ by a maximum of 10%; preferably, they are equal. In addition, in a preferred embodiment, the size of the shell area between guide tube and liquid surface and/or the shell area between guide tube and bottom of the bioreactor differ(s) by a maximum of 10% from the size of the cross sectional area of riser and/or downcomer. In a particularly preferred embodiment of the method according to the invention, the flow cross section for the circulating flow in all regions of the reactor is virtually equal or equal, in order to reduce pressure drops.

In a further preferred embodiment, the shell area between guide tube and bottom of the bioreactor is less than the cross sectional areas of riser and downcomer. In the bottom region, an increased flow velocity is thereby generated which effectively prevents sedimentation of cells or microorganisms. Preferably, the shell area between guide tube and bottom of the bioreactor is smaller by at least 5% and by a maximum of 50%, particularly preferably smaller by at least 5% and smaller by a maximum of 30%.

Cultures which can be used in the method according to the invention are microorganisms and also animal, plant and human cells.

The advantages of the invention are:
Preexisting bioreactors having a ratio of height to diameter of, for example, 2 can be simply converted to operation as an air-lift bioreactor. Expensive new capital investment is dispensed with.
Air-lift bioreactors having a low ratio of height to diameter have, not least due to a less pronounced hydrostatic pressure profile, a higher homogeneity with respect to dissolved oxygen, dissolved carbon dioxide and pH (for instance, high slender bioreactors are susceptible to local (dependent on height) carbon dioxide partial pressures which act in each case on the pH). The probability of undersupplying the cells with dissolved oxygen in the downcomer of the air-lift bioreactor falls. The generally better axial mixing also leads to improved homogeneity in the substrate concentrations.
Frequently, air-lift bioreactors have gas introduced with macrobubbles. Introducing gas with microbubbles leads to high volume-specific phase interfaces and thus makes possible a marked reduction of the gas volumetric flow rate required for driving the liquid flow. A marked reduction of the shear stress of cells compared with introducing gas as coarse bubbles is associated therewith.
The disadvantages stated for the air-lift bioreactors known from the prior art are dispensed with.

The invention will be described in more detail hereinafter with reference to figures and examples, but without restricting it thereto.

FIG. 1 shows schematically a bioreactor according to the invention (a) in cross section from the side and (b) in cross section from the top. The bioreactor according to the invention comprises a cylindrical vessel (1) in which a likewise cylindrical guide tube (2) is introduced, preferably centred in the middle. In the present example, in the guide tube, close to the bottom edge of the guide tube, a ring-shaped gas-introduction unit (3) is installed. The ratio H/D of height H to diameter D is between 1 and 6, preferably between 2 and 6.

FIG. 2 shows schematically a preferred embodiment of the bioreactor according to the invention in cross section from the top, in which the cross sectional area A within the guide tube and the area B between the outer side of the guide tube (2) and the inner wall of the vessel (1) are equal, i.e. riser and downcomer preferably possess the same size of flow cross section.

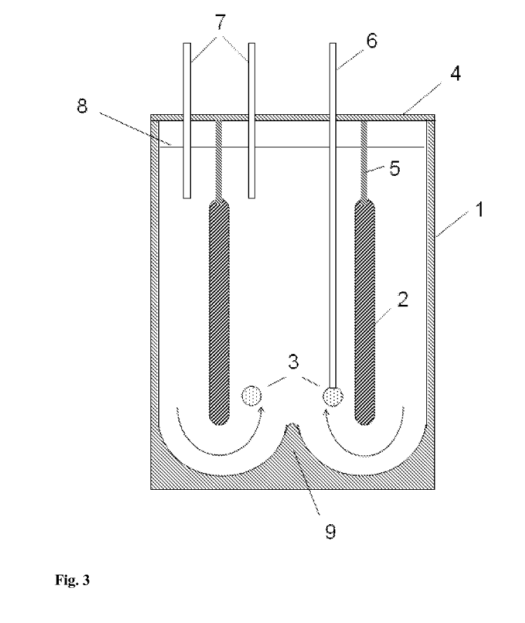

FIG. 3 shows schematically a preferred embodiment of the bioreactor according to the invention in cross section from the side. The vessel (1) preferably possesses deflecting devices (9) on the reactor bottom. The guide tube (2) is fastened to supports (5) on the lid (4) of the bioreactor. The guide tube possesses rounded edges in order to avoid pressure drops owing to eddies and shearing forces. The preferably ring-shaped gas-introduction unit, in the present example of FIG. 3, is mounted within the guide tube close to the bottom edge of the guide tube and so the riser is situated within the guide tube and the downcomer between guide tube and vessel. In addition, on the lid of the reactor, passages for the gas supply (6) and also supply of medium and/or buffer and/or additions (such as, e.g., antifoams) are mounted (7). Customarily, the bioreactor possesses means for heating and/or cooling and also sensors for measuring, e.g., temperature, pH, dissolved oxygen concentration, dissolved carbon dioxide concentration etc., which are not drawn in the present case. Preferably, the liquid level (8) in the reactor is sufficiently high that the flow cross sections in the deflector regions and in the riser and downcomer are equal.

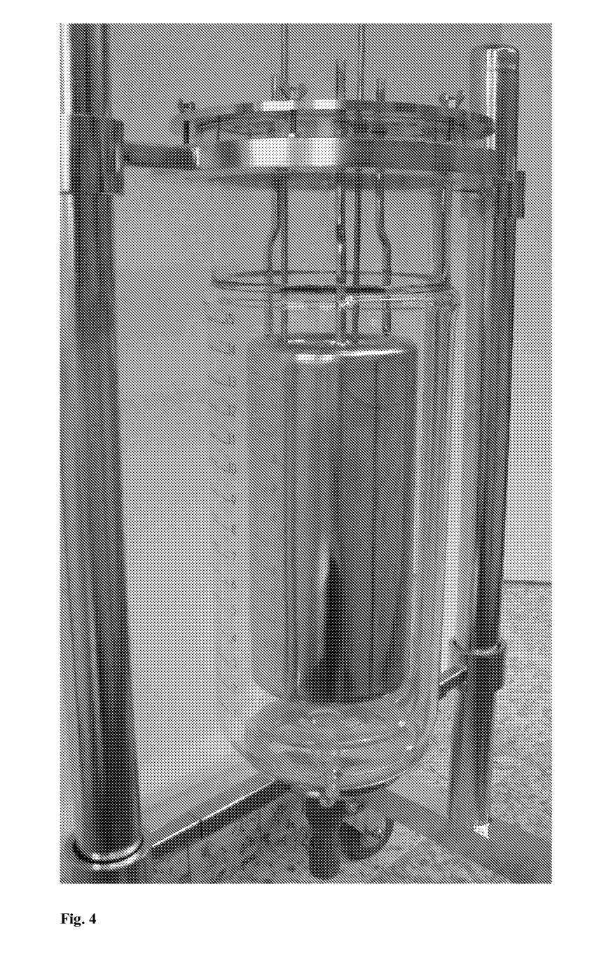

FIG. 4 shows a photograph of a preferred embodiment of the bioreactor according to the invention. The bioreactor shown comprises a glass vessel having a double shell, a lid, a bottom valve and a guide tube which can be fastened to the lid.

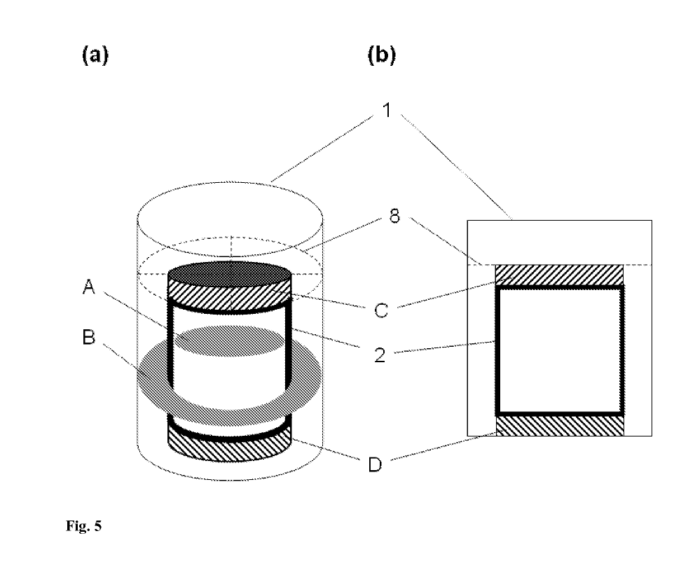

FIG. 5 shows schematically the principle of area equivalence: the cross sectional areas of the riser and downcomer and also of the shell areas above and below the guide tube are preferably equal.

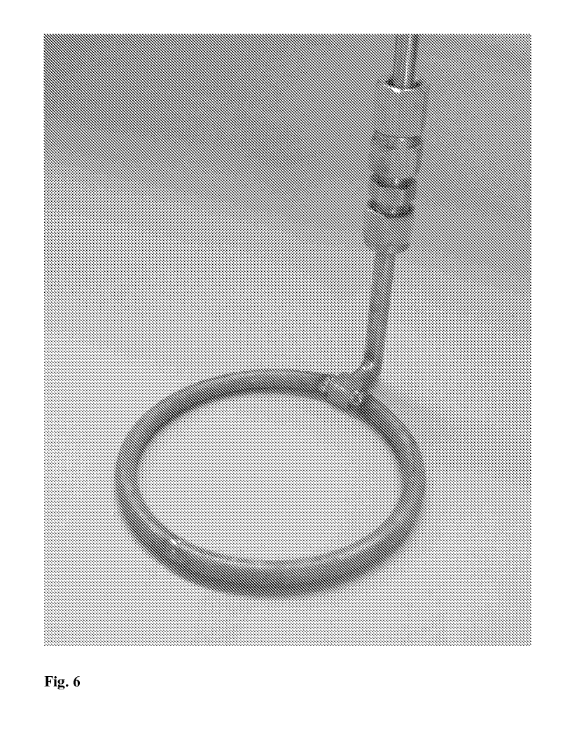

FIG. 6 shows by way of example a gas-introduction unit for the reactor according to the invention in the form of a ring-shaped microsparger.

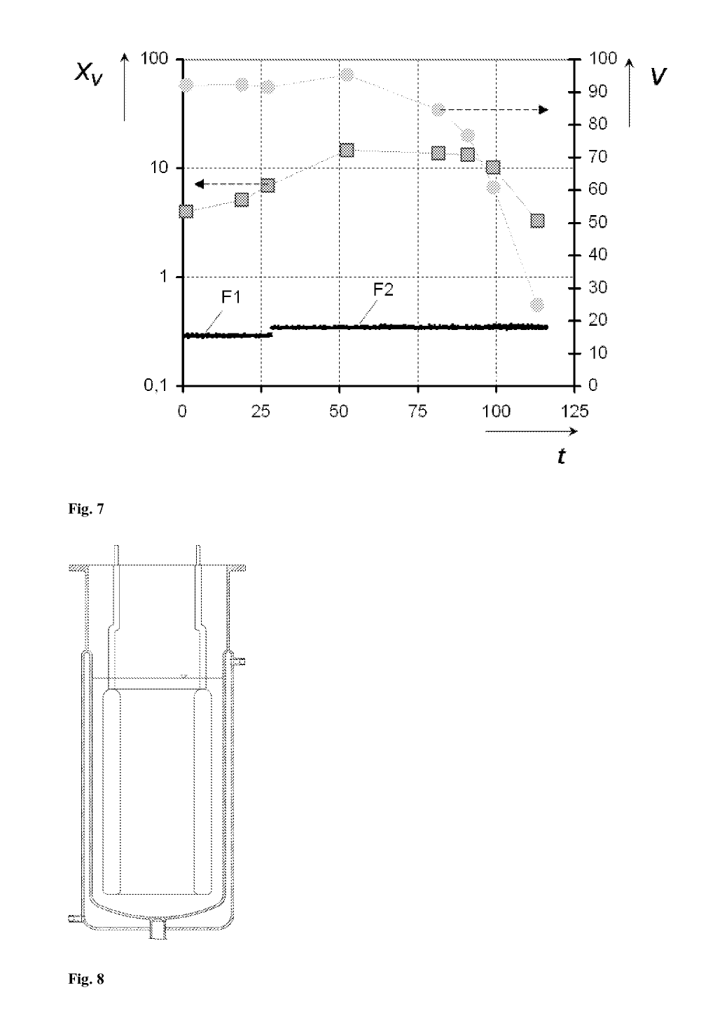

FIG. 7 shows in a graph the results of the fermentation of BHK-21 cells of Example 2 in the bioreactor of Example 1.

The live cell density X_{V} (left-hand ordinate, boxes) in the unit [10⁵ cells ml⁻¹] and the vitality V (right-hand ordinate, circles) in per cent are plotted respectively against the time t (abscissa) in hours. The time point t=0 is the time point of inoculation. In addition, the graph shows the gas-introduction rate. Gas-introduction was first started at a rate of F1=15 l/h: on the second day, the gas-introduction rate was increased to F2=17.5 l/h.

FIG. 8 serves for illustrating the data in Table 1.

Reference Signs

1 Vessel

2 Guide tube

3 Gas-introduction unit

4 Lid

5 Supports

6 Passage for gas supply

7 Passages

8 Liquid surface

9 Means for flow guidance: deflecting devices

A Cross sectional area of the riser/downcomer

B Cross sectional area of the downcomer/riser

C Shell area above the guide tube

D Shell area below the guide tube

### EXAMPLES

### Example 1

### Bioreactor

FIG. 4 shows a preferred embodiment of a bioreactor according to the invention. The bioreactor shown comprises a glass vessel having a double shell, a lid, a bottom valve and a guide tube which can be attached to the lid.

The lid boreholes are suitable for standard accessories. All components important for the later fermentation can be mounted in this manner. The tube, which acts as air feed line for the gas-introduction unit ((micro)sparger), can likewise be fastened on the lid in a height-adjustable manner. The sparger is installed centrally in the bottom part of the guide tube. By this means the ascension of the liquid flow takes place in the interior, and the descent on the exterior. The guide tube consists of a hollow double-shell cylinder. This serves not only for flow guidance; the guide tube is designed in such a manner that the installation of an internal cell separator is possible. As a result, the working volume decreases from 15 1 to 10 1.

A double shell serves for temperature control of the bioreactor in the later fermentation operation. The outflow of liquid is made possible via a bottom valve. The essential data are shown in Table 1.

TABLE 1
Design of a preferred embodiment of a bioreactor according to the invention. There is
area equivalence between the cross sectional areas of riser and downcomer and also between the
shell areas above and below the riser. The difference between maximum and actual working
volume arises owing to the guide tube, the dimensions of which serve as place holder for a
possible internal cell separator. The drawing shows the glass vessel with guide tube.

| | | |
|---|---|---|
| Maximum working volume | 0.0148 | m³ |
| H/D | 2 | |
| Cross sectional area of downcomer | 0.0110 | m² |
| = Cross sectional area of riser | 0.0110 | m² |
| Shell area below the riser | 0.0110 | m² |
| = Shell area above the riser | 0.0110 | m² |
| Actual working volume | 0.0096 | m³ |
| Diameter of riser | 0.1185 | m |
| Thickness of guide tube | 0.0280 | m |
| Distance guide tube-reactor wall | 0.0182 | m |
| Distance lower edge of guide tube-lowest point in the reactor | 0.0450 | m |
| Distance upper edge of guide tube-underneath of lid | 0.2130 | m |

The bioreactor was designed having an H/D ratio of 2. Generally, the structures of airlift fermenters are more slender—that is to say have higher H/D ratios. Inter alia, in order to avoid oxygen limitation in the downcomer, and maintain H/D ratios of common reactors, the decision was in favour of H/D=2. Table 1 likewise shows the area equivalence between the cross sectional areas of riser and downcomer and also between the shell areas above and below the riser. An equal flow velocity in all parts of the reactor results therefrom. Pressure drops and the acceleration or braking of the liquid can thus be avoided. The principle of area equivalence is shown schematically in FIG. 5.

For the gas introduction, a ring-shaped microsparger (microbubble sparger) from Mott, Farmington, Conn., USA was used, as shown in FIG. 6. In Table 2 an overview of the properties of the sparger is given.

TABLE 2
Properties of the microsparger from Mott, Farmington, CT, USA

| | Ring-shaped sparger |
|---|---|
| Pore size | 2 μm |
| Material | 316L SS |
| Description | 10.5″ sparger tube having |
| | D = 0.25″ shaped for the ring |
| | having D = 3.5″ |
| Connection | Swagelok |

### Example 2

### Fermentation for biological characterization

A BHK cell line was cultured in the bioreactor according to the invention of Example 1. BHK cells (Baby Hamster Kidney cells) are immortalized cells which were derived from the kidneys of one-day-old golden hamsters. These are fibroblasts which originally grew as adhesive. However, a multiplicity of different BHK cell lines exist, most of which have been adapted to suspension culture.

Because of their unlimited growth potential in culture, established BHK cell lines are outstandingly suitable for culture in fermenters.

In the cell culturing, a starting cell density of 4×10⁵ cells ml⁻¹ resulted having a vitality of 92%. The sparger gas introduction rate of 15 l/h was maintained at first, but increased after one day to 17.5 l/h.

During the culture, the cell density increased slightly immediately, as can be seen in FIG. 7. Within one day, the cell density doubled.

In the exponential growth phase, a growth rate of μ=0.055 h⁻¹ resulted. This is very high, compared with the growth rates in the literature. There, values between 0.02 and 0.04 h⁻¹ are cited. In the culture from which the inoculum was taken, a growth rate of 0.02 h⁻¹ was determined This deviation may be only partly explained by the uncertainty which arises from the individual measurements carried out. The high growth rate shows that the fermentation conditions can ensure optimum growth of the cells. The batch fermentation was successful under these conditions. In addition, it may observed that the formation of foam was not a significant problem. The foam, with occasional addition of antifoam C, reached a maximum height of approximately 30 mm The concentration of the antifoam, at the end of the fermentation, was approximately 40 ppm, which is an acceptable amount. For this cell line, previously, concentrations up to 500 ppm have been studied and considered not to be critical. Therefore, no foam problems arise owing to the elevated gas-introduction rate. The gas-introduction rate should, mainly for this reason, be selected to be as low as possible. Since the results indicate that the foam formation does not exceed a tolerable extent, gas can be introduced at 17.5 l/h.

1. Bioreactor constructed as an air-lift bioreactor, having a ratio H/D of height H of the bioreactor to a diameter D of the bioreactor of less than 6.

2. Bioreactor according to claim 1, wherein the ratio H/D is in the range between 2 and 6.

3. Bioreactor according to claim 1, comprising a gas-introduction unit that generates bubbles having a diameter of less than 2 mm.

4. Bioreactor according to claim 3, wherein the gas-introduction unit is a microbubble sparger.

5. Bioreactor according to claim 3, wherein the gas-introduction unit is constructed as a ring-shaped or spiral body.

6. Bioreactor according to claim 1, which comprises means at the bottom of the reactor for deflecting a flow.

7. Bioreactor according to claim 1, which comprises a riser and downcomer, wherein the cross sectional areas of riser and downcomer are equal or differ by a maximum of 10%.

8. Method for culturing microorganisms or animal or plant or human cells, comprising culturing said microorganisms or animal or plant or human cells in a bioreactor having a ratio H/D of height H to diameter D less than 6, and a loop flow (circulation flow) between an inner guide tube and a region between an outer wall of the guide tube and an inner wall of the bioreactor generated by means of a gas-introduction unit.

9. Method according to claim 8, wherein a shell area between the guide tube and liquid surface and the shell area between guide tube and bottom of the bioreactor are equal or differ by a maximum of 10%.

10. Method according to claim 9, characterized wherein the size of the shell area between guide tube and liquid surface and/or the shell area between guide tube and bottom of the bioreactor differ(s) by a maximum of 10% from the size of the cross sectional area of riser and/or downcomer.

11. Method according to claim 9, wherein the shell area between guide tube and bottom of the bioreactor is less than the cross sectional areas of riser and downcomer.

12. (canceled)
